(19) United States
(12) Patent Application Publication
(10) Pub. No.: US 2010/0254958 A1
(43) Pub. Date: Oct. 7, 2010

(54) Antigen-Specific T-Cell Preparations from Bone Marrow

(76) Inventors: Anne Letsch, Berlin (DE); Carmen Scheibenbogen, Berlin (DE); Il-Kang Na, Berlin (DE); Hans-Dieter Volk, Berlin (DE)

Correspondence Address: LERNER GREENBERG STEMER LLP, P O BOX 2480, HOLLYWOOD, FL, 33022-2480, US

(21) Appl. No.: 12/739,786

(22) PCT Filed: Oct. 24, 2008
(86) PCT No.: PCT/EP2008/009186

§ 371 Date: Jun. 18, 2010

(30) Foreign Application Priority Data
Oct. 24, 2007 (EP) 07075939.4

(51) Int. Cl. A61K 35/28; C12N 5/0783; A61P 31/12

(52) U.S. Cl. 424/93.71; 435/372.3

(57) ABSTRACT

A method for the generation of antigen-specific T-cell preparations for adoptive therapy is provided, comprising the steps of obtaining lymphoid cells from the bone marrow of a patient in a first step, expanding the lymphoid cells in cell culture medium ex-vivo in the presence of at least one of IL2 and IL7 and or more antigens, yielding a T-cell preparation, and isolating the T-cell preparation from the culture medium in an isolation step. T-cell preparations provided according to the inventive method, and the use of such T-cell preparations for the treatment of infectious disease and cancer is also provided.

An important objective of research in immunology is the generation of antigen-specific T-cells in vitro for the treatment of severe viral infections (“adoptive therapy”). The goal of adoptive therapy is the induction of an effective immune response against encountered antigen, resulting in elimination of the disease.

Adoptive cell transfer of donor-derived, ex-vivo primed and expanded human cytotoxic T lymphocytes (CTLs) has emerged as a promising approach to treat both infectious and malignant diseases in humans. CMV disease is a major cause of morbidity and mortality in immunocompromised individuals. First clinical studies have shown that transfer of Cytomegalovirus (CMV)-specific CD8+ T-cells is safe and effective in reconstitution of cellular immunity against CMV disease (Walter 2005, Micklethwaite 2007). Efficacy of adoptive T-cell therapy, however, is limited by the numbers of CTLs that can be obtained in vitro and the survival and function after infusion.

Detailed analysis of the cellular immune response has been greatly facilitated by the ability to characterize different T-cell populations phenotypically by differentiation-associated surface-markers and functionally by their secretion of different cytokines. One important method for the characterization of T-cells is flow-cytometry (FACS). Thereby, immune cells are stained with antibodies that bind to cell type specific cell-surface markers, such as the “cluster of differentiation” (CD) molecules. Cells can also be stained for molecules present in the interior of a cell, allowing the characterization of cells by their production of peptide products such as interleukins or interferons.

The characterization of antigen-specific T cells by binding of antigens to their specific T-cell receptor molecule (TCR) has been enabled by the technique of tetramer staining.

The detailed analysis of phenotypical and functional characteristics of T cells enables a further division into subpopulations apart from the traditional division of T-cells into CD4-positive “helper” and CD8-positive “effector” cells. This provides a much better understanding of the role of the different T cell subpopulations in the orchestration of cellular immune responses.

Immune cells of similar phenotypical characterization by FACS or other methods may not constitute a homogeneous population. They can originate from different compartments of the body, such as peripheral blood, the intestine or the bone marrow. Traditionally, the majority of T cell analyses have been conducted on cells derived from peripheral blood, since this is the most easily accessible compartment of the body. Peripheral-blood-derived cells, however, are probably not representative for the variety of T cell responses in the body. Other relevant compartments for immune cells are the bone marrow, a major compartment for the maturation of immune cells in adult humans, or the intestine, the respiratory tract and the skin, where much of the immune response is mounted.

The current state of research of the T-cell-borne immune response is reviewed in “Effector and Memory CTL Differentiation”, Williams and Bevan, Annual Review of Immunology, 2007, Vol. 25: 171-192.

One population of cells important for the generation of long-lasting immunity are so-called “memory” T-cells. This population has been further divided into subpopulations, among them “CD45RA (−) (negative) CCR7(−) effector memory T-cells (Tem)”, CD45RA(−)CCR7(+) central memory T cells (Tcm) and CD45RA(+)CCR7(+) “naive-like” early memory T cells (Tn). Cells belonging to the central memory subpopulation are believed to be critical to mount an effective immune response upon re-encounter of antigen.

Another population of T-cells that has recently generated much interest are multifunctional T-cells, which secrete interferon gamma (IFN-γ), tumor necrosis factor alpha (TNF) and interleukin-2 (IL-2) simultaneously. Recent studies have shown that multifunctional CD4(+) T-cells simultaneously secreting IFN-γ, TNF and IL-2 correlated best with protection against leishmania major (Darrah et al., Nature Med. 2007, 13, 843-850) and with superior functionality against CMV (Kannanganat et al., J Virol. 2007, 81, 8468-8476).

One objective of the present invention is to provide methods for the generation of T-cell preparations for adoptive therapy that enable the generation of an efficient and lasting immune response against a disease-related antigen. A further objective of the present invention is to provide such preparations in the form of a preparation of antigen-specific T-cells, and a method for their use in the treatment of disease.

These objectives are attained by the methods and cell preparations according to the independent claims.

In the present document, “CD45RA” means the human naive T lymphocyte marker and CCR7 means the human chemokine receptor 7.

If any cell population is designated “positive” with respect to a certain marker protein, this designation shall mean that said cell population can be stained by a common fluorescent-dye-labelled antibody against the marker protein and will give a fluorescence signal of at least one log higher intensity compared to unlabelled cells or cells labelled with the same antibody but commonly known as not expressing said marker protein.

A cell designated as “tetramer positive” is one that shows selectivity against a known antigen fragment in an assay known in the art as tetramer staining.

Methods to perform antibody or tetramer labelling and fluorescence measurement are known in the art and well documented in scientific publications such as “Methods”.

In the course of our investigations, we have analysed whether bone marrow is superior to peripheral blood for expansion of antigen-specific T-cells. T-cells specific for known antigens of cytomegalovirus (CMV) were employed as an example. CMV is a clinically relevant pathogen, to which many human patients acquire immunity in the course of their lives. The general principle of T-cell generation presented here, however, is applicable to a number of antigens that are operational within the context of a T-cell borne immune response.

Other antigens or epitopes to which the current invention is applicable include antigens of Ebstein Barr virus (EBV), human papillomavirus (HPV), human immunodeficiency virus (HIV), or tumour-associated antigens such as WT1, PAX2/8, Tyrosinase, and/or MAGE.

Hence, the present invention shall not be limited to CMV-specific T-cells but is rather directed towards a general method of providing pathogen-specific cell preparations for use in medicine.

In a first study the phenotype and function of CMV-pp65 specific CD8+ T-cells in normal human peripheral blood and bone marrow was compared using intracellular cytokine and tetramer staining. It was found surprisingly that the frequency of CD45RA(−) CCR7(+) central memory T-cells (Tcm) was significantly higher in bone marrow compared to peripheral blood (p<0.01).

Only from bone marrow, CMV-specific CD45RA(−) CCR7(+) central memory T-cells (Tcm) could be expanded.

CMV-pp65 specific CD8(+) T-cells could expanded be more efficiently from bone marrow (mean 109-fold, n=6) than from peripheral blood (mean 74-fold, p=0.01) after 14 days in vitro culture.

Methods of expansion are shown in example 2

In a second study, the expansion of CD4(+) CMV-specific T-cells from bone marrow was studied. CD4(+) T-cells are considered to play an important role in maintaining a long-lasting immune response. Both frequencies and cytokine co-expression profiles of expanded T-cells specific for the CMV proteins IE1 and pp65 (tested against a pool of overlapping peptides spanning the whole protein) were evaluated. These proteins comprise among the most frequently recognized epitopes.

Our results demonstrate that the bone marrow contains higher frequencies of CMV-specific CD45RA(+)CCR7(+) (“naive”) and CD45RA(−)CCR7(+) “central memory” T cells than peripheral blood. Furthermore these results demonstrate that
1) total CD4(+) and CD8(+) T cells have a higher proliferative capacity in bone marrow.
2) CMV-specific CD4(+) T cells can be expanded more efficiently from bone marrow and
3) Within the expanded CMV-specific CD4(+) T cells a higher proportion is multifunctional (IFNγ(+), TNF(+) and IL-2(+)) in bone marrow than in peripheral blood.

The degranulation potentials in CD4(+) and CD8+ T-cells in bone marrow and peripheral blood are similar.

Paired peripheral blood and bone marrow samples were obtained from patients who underwent total hip arthroplasty. T-cells were expanded in the presence of IL2 and IL7 either from bulk culture with exposure to two different peptide pools (IE1 and pp65), or after selection via IFN-γ secretion by stimulation with pp65. CMV specific immune responses were assessed by using multiparameter flow cytometry staining cells for CD3, CD4, CD8, CCR7 and CD45RA and for the cytokines IFN-γ, IL2 and TNF-α at day 0 and after 10 days of in vitro expansion (see example 2).

Similar frequencies of cytokine-producing pp65- and IE1-specific T-cells were found in unmanipulated paired peripheral blood and bone marrow samples. Expansion of CMV-specific T-cells from bone marrow resulted in significantly higher frequencies of antigen-specific CD4(+) T-cells than from peripheral blood.

Significantly higher frequencies of bone marrow pp65 and IE1-specific CD4(+) T-cells were multifunctional, characterized by producing simultaneously IFN-γ, TNF and IL-2 (IE1: bone marrow mean 0.44%±0.216; peripheral blood mean 0.109%±0.105, p=0.031; pp65: bone marrow mean 3.987%±2.546; peripheral blood mean 1.24%±0.90, p=0.031). Expansion of multi-functional CD4(+) T-cells from bone marrow was observed with both the bulk and selection assay protocol. Both peripheral blood and bone marrow CMV-specific CD4(+) and CD8+ T-cell lines had a predominant CD45RA− CCR7− effector memory phenotype.

According to one aspect of the present invention, antigen-specific adoptive T-cell immunotherapy is provided by obtaining T-cells from the bone marrow, expanding the cells in-vitro (ex-vivo) in the presence of the specific antigen against which the therapy is directed, and returning the expanded T-cells into the patient.

According to one aspect, the current invention provides a method for the generation of preparations of T-cells that are specific at least one target antigen. Such preparation can be advantageously be used in adoptive transfer for medical purposes, particularly in the treatment of infectious diseases, cancer and other diseases where antigen-specific T-cells is of benefit to the patient. The method according to the invention comprises the steps of obtaining lymphoid cells from the bone marrow of a patient in a first step, expanding said lymphoid cells in a cell culture medium comprising at least one of interleukin 2 and interleukin 7 ex-vivo in an expansion step, yielding a T-cell preparation, and isolating the T-cell preparation from the cell culture medium in an isolation step.

The isolation step by way of example may comprise sedimenting the cell culture by centrifugation or other means to concentrate the cell preparation, so as to facilitate the application of the inventive cell preparation to a patient, for example by injection.

According to one aspect of the invention, a method is provided as described in the preceding two paragraphs, whereby during the expansion step, one or more target antigens or peptide fragments thereof are present in the cell culture medium. Such target antigen presence, for example in soluble form as peptides or protein fragments, or complexed to carrier structures, will stimulate the cells present in the culture medium that have a natural propensity to react to the presence of such target antigen, thus stimulating the cells that are able to react, and increasing their presence in the culture.

According to yet another aspect of the invention, a method is provided as described in the preceding paragraphs, whereby after the first step, the lymphoid cells are cultured in a cell culture medium ex-vivo in the presence of one or more antigens in a stimulation step. Subsequently, cells that secrete at least one of IFN-γ, TNF-a and IL-2, and/or are tetramer positive with respect to a target antigen, are selected in a selection step, the selected cells are separated from other cells and further submitted to the expansion step.

The selection of cells according to their cytokine expression profile may help to bias the population during its culture to develop a stronger functional bias. Similarly, the selection of cells that are tetramer-positive with respect to target antigens selects for cells that have a natural ability to react to the target antigen, again providing bias in the development of the population and creating a stronger immune message and effect upon eventual re-introduction into the patient's body.

According to yet another aspect of the invention, a method is provided as described in the preceding paragraphs, whereby the expansion step is performed at least 7 days. Longer expansion steps may enable the growth of larger target-antigen-specific populations still, leading to more robust reactions upon re-introduction into the patient. For example, expansion steps may last 10, 20 or 30 days, and may be extended beyond that measure.

According to one aspect of the current invention, cells prepared by the methods described herein may be frozen and stored, for example between the first step and the expansion step.

According to yet another aspect of the invention, the isolation step may comprise or be followed by at least one final selection step selecting for cells either positive or negative for CD45RA, and/or selecting for cells either positive or negative for CCR7. Hence, depending on the criteria for selection, such final selecting step may isolate the following populations:
CD45RA(+), CCR7(+) cells which had no antigen contact yet. A small proportion of these T cells, which could be detected by cytokine production in response to brief peptide stimulation, are functionally not naïve, but represent most likely a less differentiated memory subset.

CD45RA(−), CCR7(+) cells remain present in the absence of antigen stimulation and have a higher proliferative potential and greater capacity than effector memory T cells for long-lasting persistence in vivo. They can recirculate more easily to T cell zones of peripheral lymphoid tissue, take longer than effector memory T cells to differentiate into effector T cells.
CD45RA(−), CCR7(−) cells remain present in the absence of antigen stimulation and can rapidly mature into effector T cells and secrete large amounts of cytokines after antigen stimulation.
CD45RA(+), CCR7(−) cells are found in the setting of active antigen stimulation, able to eliminate viruses and tumors by different effector functions.

According to yet another aspect of the invention, a method is provided as described above, whereby the target antigens comprise CMV antigens, particularly peptides representing CMV antigens pp65 and IE1. Alternatively or additionally, the target antigens may be tumor-associated antigens, particularly peptides of WT1, MAGE, PAX2/8, Tyrosinase, MAGE and/or Epstein-Barr-virus-associated antigens.

Another object of the invention is a T-cell preparation that can be obtained by a method according to any of the aspects of the invention laid out above.

According to one aspect of the invention, such preparation comprises T-cells specific for a target antigen, particularly T-cells specific for CMV antigens. Of these target-antigen-specific T-cells, between 1% and 10% are CD4(+) CD45RA positive, CCR7 positive naïve-like early memory cells, between 10% and 20% are CD4(+) CD45RA negative, CCR7 positive central memory cells, between 60% and 90% are CD4(+) CD45RA negative, CCR7 negative effector memory cells, and/or between 1% and 4% are CD4(+) CD45RA positive, CCR7 negative EMRA cells.

The T-cell preparation for adoptive transfer thus provided can be used as a medicament in human patients, as a treatment against an infectious disease, exemplified by its use against infection by cytomegalovirus. Similarly, a T-cell preparation can be employed in the treatment of other viral infections or a malignant tumour disease.

The invention is further illustrated by the following figures and examples.

### DESCRIPTION OF FIGURES

FIG. 1 shows the result of multiparameter flow cytometry determining the total frequency of IFN-γ, TNF and IL-2 producing CD4(+) and CD8(+) cells at day zero (A), previous to expansion and at day 10 (D). The mean is shown +−s.e.m.(B) illustrates the expansion factors of total CD4(+) and CD8(+) T cells in paired peripheral blood/bone marrow samples, shown are the individual patients (squares) and bars indicate the median value of the group. The median absolute number is shown in (C). Y-axes are % Cytokine-producing cells (A, D), Expansion factor (B) and 10E5 cells (C).

FIG. 2 shows higher frequencies of CD4(+)cytokine producers in bone marrow vs peripheral blood. The y-axis shows cytokine-producing cells in %.

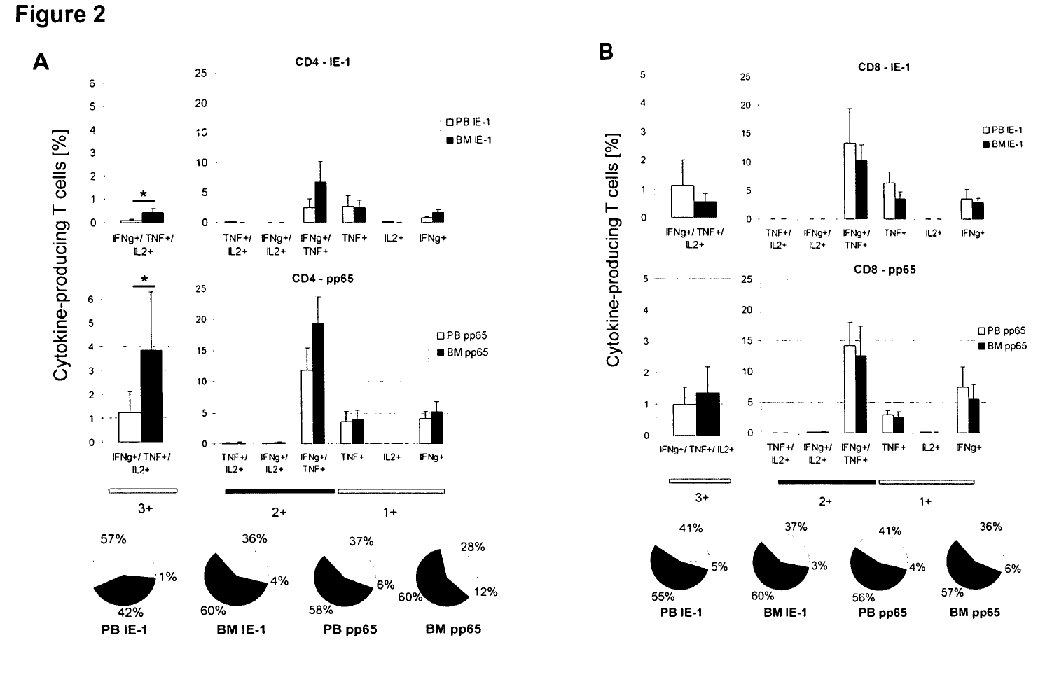

FIG. 3 shows higher cytokine production per cell in triple vs single cytokine+ CD4 T cells. The Y-axis shows the mean fluorescence intensity.

FIG. 4 shows higher frequencies of CCR7+ CMV specific T cells in bone marrow at day 0 and dominant effector/memory phenotype in both compartments at day 10. The y-axis shows % of CMV-specific T-cells.

FIG. 5 shows comparable cytotoxic capacity of CMV-specific T cells from bone marrow and peripheral blood as analysed by CD107a mobilization. The y-axis shows the number of CD107(+) cells in %.

### EXAMPLES

### Example 1

### Peripheral Blood and Bone Marrow Samples

PBMC were obtained from autologous blood banking of patients undergoing total hip arthroplasty, and paired BM samples were collected from resected femoral heads. Peripheral blood and bone marrow mononuclear cells were isolated by density gradient centrifugation using Ficoll-Hypaque and cryopreserved until analysis. All analyses performed in our studies were done with cryopreserved samples. Fresh samples would, however, also be suitable for T-cell generation.

### Example 2a

### Bulk Expansion Protocol

Synthetic CMV peptides: Standard 15-amino acid peptides (11 overlaps) spanning the CMV IE-1 and pp65 proteins were purchased from JPT peptide technologies, Berlin. The peptides were dissolved in DMSO (Merck, Darmstadt, Germany). Peptide sequences were employed as described in Cherepnevet al, Use of peptides and peptide libraries as T-cell stimulants in flow cytometric studies, Methods Cell Biol. 2004; 75: 453-79.

Cells were thawed and resuspended in RPMI Medium (Biochrom, Berlin, Germany) containing 4% (vol/vol) AB serum (Valley Biomedical, Winchester, USA), 2% (vol/vol) L-glutamine (PAA Cölbe, Germany), and 1% (vol/vol) penicillin/streptomycin (PAA, Cölbe, Germany). After overnight incubation with Iscoves Medium (Biochrome Berlin, Germany) containing 10% AB Serum at 37° C. in a humidified 5% CO₂ atmosphere, mononuclear cells from peripheral blood or bone marrow samples were then cultured in 96-well round-bottom plate (2×10⁵ cells per well) together with CMV peptide pools (IE-1 or pp65, c=1 μg/ml each peptide, JPT Peptides Technologies, Berlin, Germany), rhIL-2 (50 IU/ml, R&D Systems, Wiesbaden, Germany) and rhIL-7 (10 ng/ml, R&D Systems). Media and IL-2 were renewed on days 3 and 5. IL-7 was added weekly. After 10 days of culture, cells were harvested, washed, and co-stained with IFNγ, TNFα and IL-2 for quantitation of CMV-specific T-cells.

### Example 2b

### Expansion of Interferon (IFN)-γ Secreting Cells (Selection Protocol)

Cells were thawed and incubated overnight at a cell concentration between 1×10⁶ cells and 2×10⁶ per ml per well of a 24-well Plate (Costar, Corning Incorporated, Corning N.Y., USA). After incubation cells were stimulated with CMV peptide pools (pp65, JPT Peptides Technologies, Berlin, Germany). The final concentrations of (each) peptide were 1 μg/ml. The IFN-γ cytokine secretion assay was performed by magnetic columns according to the manufacturer's instructions (Miltenyi Biotech, Bergisch-Gladbach, Germany). Enriched cells were seeded in 96-well flat-bottom plate (if possible 2-5×10⁴ cells per well) in the presence of 5-10×10⁶ irradiated autologous PBMC in complete Medium supplemented with 100 U/ml rhIL-2 (Proleukin, Chiron) and 10 ng/nl rhIL-7 (ImmunoTools, Friesoythe, Germany). The medium was changed on day 4 and then every week two to three times. After formation of confluent T-cell layers, cells were divided 1:2. FACS analysis was performed at least after 14 days.

### Example 3

### Phenotype Determination

Interferon (IFN)-γ secretion assay: Cells were thawed and incubated overnight at a cell concentration between 1×10⁶ cells and 2×10⁶ per ml per well of a 24-well Plate (Costar, Corning Incorporated, Corning N.Y., USA). After incubation cells were stimulated with CMV peptide pools (IE-1 or pp65, JPT Peptides Technologies, Berlin, Germany). The final concentrations of (each) peptide were 1 μg/ml. The IFN-γ cytokine secretion assay was performed according to the manufacturer's instructions (Miltenyi Biotech, Bergisch-Gladbach, Germany). Enriched cells were seeded in 96-well flat-bottom plates (if possible 2×10⁴-5×10⁴ cells per well) in the presence of 5-10×10⁶ irradiated autologous PBMC in complete Medium supplemented with 100 U/ml rhIL-2 (Proleukin, Chiron) and 10 ng/nl rhIL-7 (ImmunoTools, Friesoythe, Germany). The medium was changed on day 4 and then every week two to three times. After formation of confluent T-cell layers, cells were divided 1:2. FACS analysis was performed at least after 14 days (see following paragraphs).

Multiparameter flow cytometry: T-cell analyses were performed simultaneously from peripheral blood and bone marrow at day 0 and after 10 days expansion. Following thawing of cryopreserved samples and overnight resting, antigen-specific T-cells were detected by intracellular TNF, IFN-γ and IL2 accumulation induced by CMV peptide pools assessed by flow cytometry as described previously (Asemissen et al., Clin. Canc. Res, 2006, 12:7476-82). In brief, 1-2×10⁶mononuclear cells were stimulated for 6 hours with IE-1 or pp65 peptide pools (1 μg/ml) and DMSO as negative control. After 1 hour, 10 μg/ml brefeldin A (Sigma, Deisenhofen, Germany) was added and after additional 5 hours, cells were stained with fluorescence-conjugated mAb against CD3, CD4, CD8, CD45RA (Beckman Coulter, Krefeld, Germany), CCR7 (R&D, +anti-IgG2A-biotin (Southern Biotech/Biozol, Eching, Germany)+Biotin-Streptavidin (Invitrogen Paisley, UK)) and IFN-γ, IL-2 and TNF. All antibodies and reagents for intracellular cytokine staining were purchased from BD Pharmingen except where noted. Positive events counted in response to incubation with DMSO were subtracted from the response obtained by specific stimulation. Data acquisition was performed on BD LSRII flow cytometer and analyzed using Cellquest and Diva software (BD Biosciences).

Ex vivo CD107 labelling: For the analysis of degranulation and co-stimulatory potential, cells were pre-stained with anti-human CD107a-PE (BD Biosciences Pharmingen). Lymphocytes were then stimulated in vitro with peptide pools in the presence of this antibody for 5 h at 37° C. in a humidified 5% CO2 atmosphere. After 1 h 15 μl/ml monensin (2 mM stock solution) was added. Cells were then harvested, washed, and stained for other surface molecules and fixed and permeabilized and stained for intracellular cytokines. DMSO without any addition of peptides was included as a negative control for spontaneous CD107a expression and/or cytokine production.

Statistics: All comparisons of T-cell frequencies and phenotype in peripheral blood and bone marrow were performed using a two-tailed Wilcoxon matched pairs signed rank sum test to calculate the p values. For the main hypothesis that there are significant more multifunctional CD4(+) T-cells expandable using BM as a source in comparison to peripheral blood, p-values less than 0.05 were considered significant. P-values for additional comparisons are considered exploratory because of multiple testing. All cytokine frequencies reported are after subtraction of background frequencies in the absence of peptides. Calculations were performed using SPSS software (SPSS inc.).

### Comparative Example 4

### Frequencies of CMVpp65 and IE1-Specific CD4(+) and CD8(+) T-Cells in Peripheral Blood and Bone Marrow

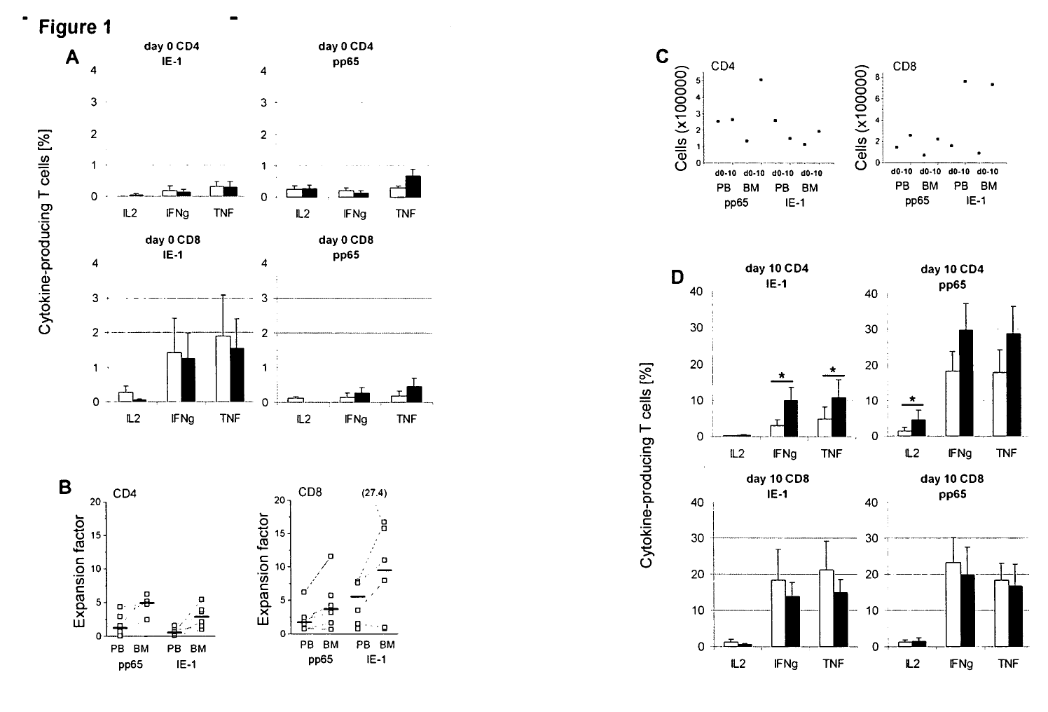

CMV pp65/IE1-specific T-cell responses were quantitated simultaneously in peripheral blood and bone marrow from 6 subjects using IFN-γ/TNF-α/IL-2 cytokine staining. Similar frequencies of cytokine-producing pp65- and IE1-specific CD4(+) and CD8(+) T-cells were found in unmanipulated paired peripheral blood and bone marrow samples (see FIG. 1).

### Example 5

### Expansion of CMV-Specific T-Cells From Peripheral Blood Versus Bone Marrow

To examine expansion potential of pp65 and IE1 CMV-specific T-cells in peripheral blood vs. bone marrow, paired peripheral blood and bone marrow samples from 6 subjects were simultaneously cultured for 10 days in the presence of specific peptides (IE1, pp65), IL-2 and IL-7. Expansion of CMV-specific T-cells from bone marrow resulted in significantly higher frequencies of specific CD4(+) T-cells than from peripheral blood after 10 days (frequency of total specific CD3+CD4(+) T-cells (IE1+pp65): bone marrow mean 22.08%±5.74, peripheral blood mean 12.96%±4.17, p=0.017, one-sided). Considering single cytokine and single peptide pool, there were significant differences for CD4(+) T-cells for IFN-γ [IE1] and TNF-α [IE1] (p=0.016, one-sided), and for IL-2 [pp65] (p=0.031, one-sided).

Differentiation Phenotypes of Total and CMV-Specific T-Cells in Peripheral Blood and Bone Marrow

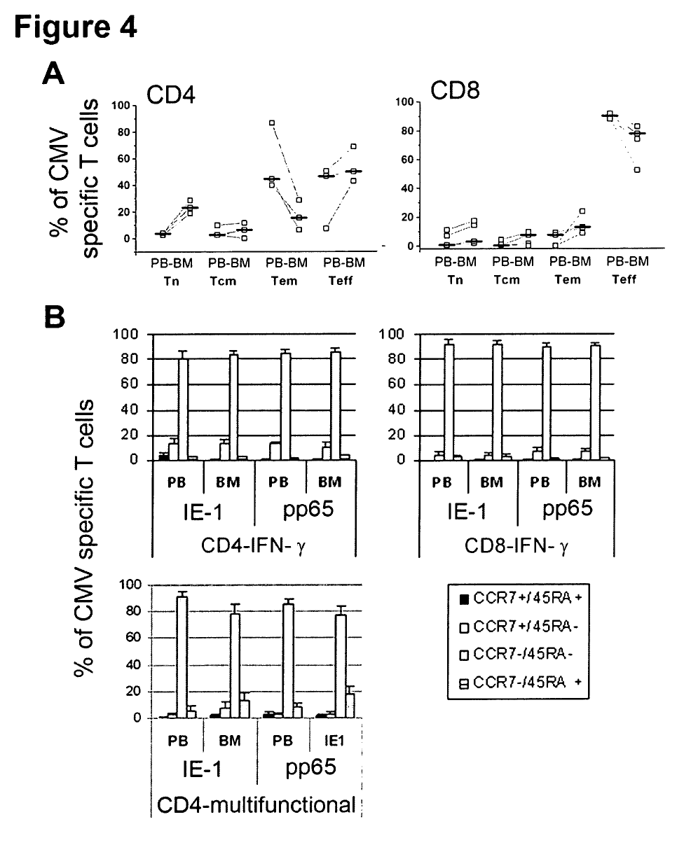

The classification of T-cell-differentiation subsets is based on the expression of CCR7 and CD45RA as described previously (Sallusto et al., Nature 1999, 401, 708-712). (N=naïve, CM=central memory, EM=effector memory, EMRA=effector). Phenotypic characteristics of CMV-specific bone marrow/peripheral blood T-cells were distinct at day 0 with more CCR7+CD45RA+ and CCR+CD45RA− in BM (see FIG. 4A). After 10 days expansion, both CMV-specific total CD4(+) and CD8(+) T-cells had a predominant CD45RA−CCR7− effector memory phenotype in peripheral blood and bone marrow (FIG. 4B).

### Example 6

### Multifunctional CMV-Specific T-Cells can be Expanded from Bone Marrow with High Efficacy

To further define the quality of T-cell cytokine response, all combinations of IFN-γ, TNF-α and IL-2 at the single-cell level were assessed by multiparameter flow cytometry and categorized the cytokine-positive cells into seven different subsets consisting of triple producers, double producers, and single producers (FIG. 1). Thus the total frequency of specific T-cells encompasses following distinct populations: IFN-γ single-positive, TNF-α single-positive, IL-2 single positive, IFN-γ+TNF-α+, IFN-γ+IL-2+, TNF-α+IL-2+ and IFN-γ+TNF-α+IL-2+. Simultaneous production of all three cytokines could lead to better expandable and more efficient T-cells.

Significantly higher frequencies of bone marrow pp65 and IE1-specific CD4(+) T-cells were multifunctional when T-cell preparations were prepared from bone marrow compared to cell originating in peripheral blood. These multifunctional cells were characterized by producing simultaneously IFN-γ, TNF-α and IL-2 (IE1: bone marrow mean 0.4%±0.2; peripheral blood mean 0.1%±0.1, p=0.031; pp65: bone marrow mean 3.9%±2.5; peripheral blood mean 1.2%±0.9, p=0.031).

Expansion of CMV-Specific T-Cells Selected by IFN-γ Secretion Assay

Expansion of multi-functional CD4(+) T-cells from bone marrow was observed with both the bulk and selection assay protocol.

Cytokine-Production Per Cell in Peripheral Blood and Bone Marrow

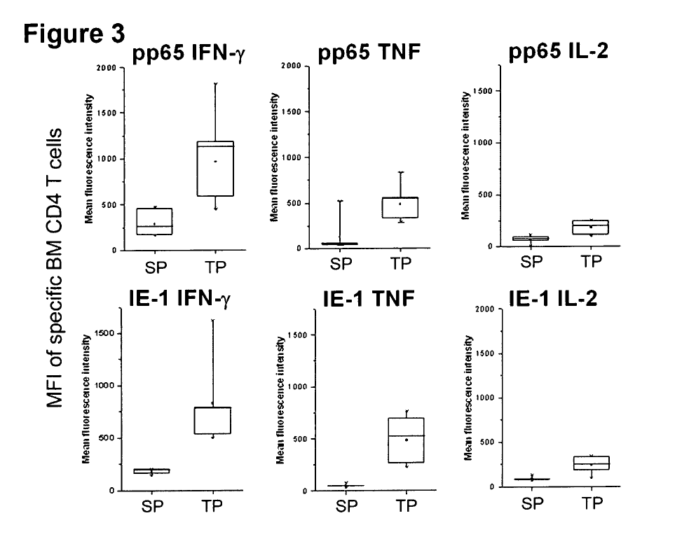

Another mechanism by which cells could mediate enhanced protection is the possibility that T-cells produce more cytokines on a per-cell basis. The mean fluorescence intensity (MFI) of single (SP) and triple (TP) cytokine producing bone marrow derived CD4(+) T cells specific for pp65 and IE-1 was determined. Triple cytokine producing CMV-specific CD4(+) T cells exhibited a 2- to 4-fold increased MFI (p<0.05) for IFN-γ, TNF and IL-2 compared to the respective single producers (FIG. 3).

Cytotoxic Potential of CMV-Specific T-Cells

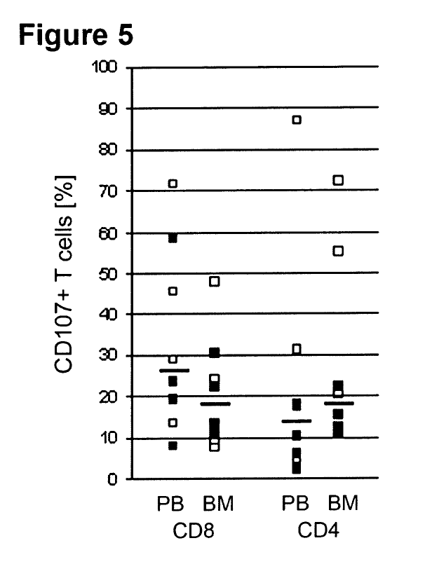

We investigated whether cell subsets and cell populations from other origins (peripheral blood and bone marrow) differed in their ability to degranulate following stimulation and thereby might differ in their cytolytic potentials. The degranulation potential was measured as the surface mobilization of CD107a, which has been shown to be associated with cytolytic potential in CD8 (Betts 2003) and CD4 T-cells. We studied 4 paired bone marrow and peripheral blood CMV-specific T cell lines to determine degranulation in response to stimulation with IE-1 and pp65 peptide pools. There were slightly lower frequencies of CD107+CD8(+) and higher frequencies of CD107+CD4(+) in bone marrow as compared to PB (FIG. 5). As CD107a T cell frequencies corresponded to the cytokine-producing CMV-specific T cell frequencies in peripheral blood and bone marrow, these findings reveal a comparable cytotoxic potential of peripheral blood and bone marrow CMV-specific T cells.

1-10. (canceled)

11. A method for generating preparations of T-cells that are specific at least one target antigen, for use in adoptive transfer, the method which comprises the following steps:
obtaining lymphoid cells from the bone marrow of a patient in a first step;
culturing the lymphoid cells in a cell culture medium ex-vivo in the presence of one or more antigens in a stimulation step following the first step;
subsequently selecting cells that
secrete at least one of IFN-gamma, TNF-a and IL-2; and/or
are tetramer positive with respect to a target antigen;
separating the selected cells from other cells in a selection step;
subsequently expanding the lymphoid cells in a cell culture medium containing at least one of interleukin 2 and interleukin 7 ex-vivo in an expansion step, yielding a T-cell preparation; and
isolating the T-cell preparation from the cell culture medium in an isolation step.

12. The method according to claim 11, wherein, during the expansion step, one or more target antigens or peptide fragments thereof, are present in the cell culture medium.

13. The method according to claim 11, wherein the expansion step is performed at least 7 days.

14. The method according to claim 11, wherein the isolation step comprises or is followed by at least one final selection step selecting for cells either positive or negative for CD45RA, and/or selecting for cells either positive or negative for CCR7.

15. The method according to claim 11, wherein the target antigens comprise CMV antigens and/or tumor-associated antigens.

16. The method according to claim 15, wherein the CMV antigens are peptides representing CMV antigens pp65 and IE1.

17. The method according to claim 11, wherein the tumor-associated antigens are peptides of WT1, MAGE, PAX2/8, Tyrosinase, MAGE and/or Epstein-Barr-virus-associated antigens.

18. A T-cell preparation, obtained by the method according to claim 11.

19. A T-cell preparation obtained by the method according to claim 11, wherein the preparation comprises target-antigen-specific T-cells, of which between 60% and 90% are CD4(+) CD45RA negative, CCR7 negative effector memory cells, and
between 1% and 10% are CD4(+) CD45RA positive, CCR7 positive naïve-like early memory cells
between 8% and 20% are CD4(+) CD45RA negative, CCR7 positive central memory cells, and/or
between 1% and 5% are CD4(+) CD45RA positive, CCR7 negative EMRA cells.

20. A T-cell preparation according to claim 18, comprising at least 0.1% of cells that are positive with regard to target-antigen-specific tetramer-staining and/or secrete at least one of IFN-γ, TNF-α and IL-2.

21. A method of preparing a medicament, which comprises preparing a T-cell preparation with the method according to claim 11 and using the T-cell preparation for the preparation of the medicament.
